(19) United States
(12) Patent Application Publication
(10) Pub. No.: US 2024/0302307 A1
(43) Pub. Date: Sep. 12, 2024

(54) SENSOR MODULE, CONTROL APPARATUS, CONTROL METHOD, AND CONTROL PROGRAM

(71) Applicant: ROHM Co., LTD., Kyoto (JP)

(72) Inventor: Shunsuke Akasaka, Kyoto (JP)

(21) Appl. No.: 18/597,075

(22) Filed: Mar. 6, 2024

(30) Foreign Application Priority Data
Mar. 9, 2023 (JP) 2023-036285

(51) Int. Cl. G01N 25/18; G01N 25/56

(52) U.S. Cl. CPC G01N 25/18; G01N 25/56

(57) ABSTRACT

Provided is a sensor module that measures concentration of at least one type of gas contained in a gas mixture. The sensor module includes a microheater of thermal conduction type that generates heat according to supplied power, and a control apparatus that is able to communicate with the microheater, in which the control apparatus detects a first detection value corresponding to first temperature of the microheater at a time of supply of first power, detects a second detection value corresponding to second temperature of the microheater at a time of supply of second power higher than the first power, and calculates the concentration of the at least one type of gas contained in the gas mixture based on the first detection value, the second detection value, and thermal conductivity data related to thermal conductivity of each of a plurality of types of gases contained in the gas mixture.

### CROSS REFERENCE TO RELATED APPLICATIONS

This application claims priority benefit of Japanese Patent Application No. JP 2023-036285 filed in the Japan Patent Office on Mar. 9, 2023. Each of the above-referenced applications is hereby incorporated herein by reference in its entirety.

### BACKGROUND

The present disclosure relates to a sensor module, a control apparatus, a control method, and a control program that measure the concentration of at least one type of gas contained in a gas mixture.

### DESCRIPTION OF THE RELATED ART

In the past, there is a well-known sensor that measures the concentration of at least one type of gas contained in a gas mixture. For example, a thermal-conduction gas sensor that uses the thermal conductivity of gas to measure the concentration of the gas is disclosed in Japanese Patent Laid-open No. 2017-173126.

### BRIEF DESCRIPTION OF THE DRAWINGS

FIG. 1 is a diagram for describing components of a sensor module according to a first embodiment;

FIG. 2 is a diagram for describing components of a microheater according to the first embodiment;

FIG. 3 depicts an example of the thermal conductivity of each of a plurality of types of gases;

FIG. 4 depicts an example of the thermal conductivity relative to the temperature in each of the plurality of types of gases;

FIG. 5 depicts the heater temperature relative to the heater power corresponding to the types of gases contained in a gas mixture;

FIG. 6 depicts the heater temperature relative to the heater power corresponding to the types of gases contained in the gas mixture;

FIG. 7 depicts the heater temperature relative to the heater power corresponding to the types of gases contained in the gas mixture;

FIG. 8 depicts the hydrogen concentration relative to the dew point measured by a control apparatus of a comparative example;

FIG. 9 depicts an example of simultaneous equations used by a control apparatus of the first embodiment to execute a concentration measurement process;

FIG. 10 is a flow chart illustrating the concentration measurement process executed by the control apparatus of the first embodiment;

FIG. 11 is a diagram for describing components of a sensor module and a control apparatus according to a second embodiment;

FIG. 12 is a flow chart illustrating a process of the control apparatus according to the second embodiment;

FIG. 13 is a diagram for describing components of a sensor module and a control apparatus according to a third embodiment; and

FIG. 14 is a flow chart illustrating a process of the control apparatus according to the third embodiment.

### DETAILED DESCRIPTION

### First Embodiment

A first embodiment of the present disclosure will be described in detail with reference to the drawings. Note that the same reference signs are provided to the same or corresponding parts in the drawings, and the description will not be repeated.

### [Configuration of Sensor Module]

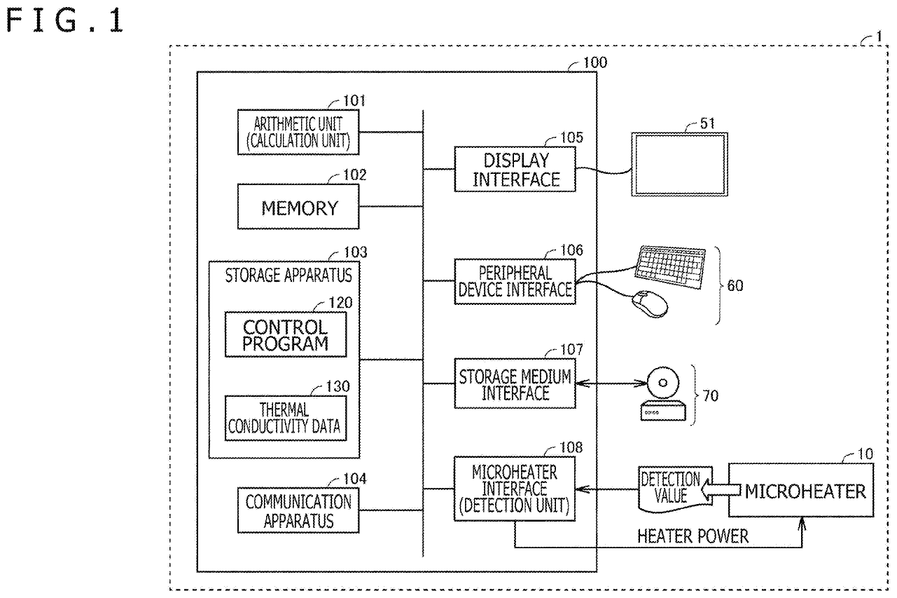

Components of a sensor module 1 according to the first embodiment will be described with reference to FIGS. 1 to 4. FIG. 1 is a diagram for describing the components of the sensor module 1 according to the first embodiment. As illustrated in FIG. 1, the sensor module 1 of the first embodiment includes a microheater 10 and a control apparatus 100.

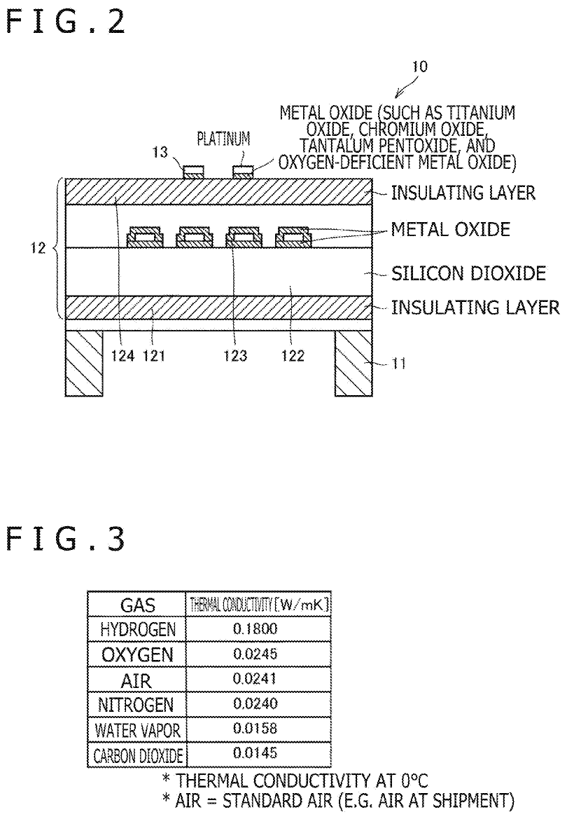

The microheater 10 is a chip gas sensor that uses MEMS (micro electro mechanical systems) technology to measure gas to be measured, such as hydrogen. FIG. 2 is a diagram for describing components of the microheater 10 according to the first embodiment. As illustrated in FIG. 2, the microheater 10 includes a silicon substrate 11, a thin-film layer 12, and at least one sensor unit 13.

The thin-film layer 12 is provided on the silicon substrate 11 and has a thickness of, for example, approximately 2.4 μm. The thin-film layer 12 includes an insulating layer 121, an intermediate layer 122 provided on the insulating layer 121 and containing silicon dioxide, at least one heater unit 123 provided in the intermediate layer 122 and containing a metal oxide, and an insulating layer 124 provided on the intermediate layer 122.

The sensor unit 13 is provided on the insulating layer 124 of the thin-film layer 12, and the sensor unit 13 contains a metal oxide and platinum provided on the metal oxide. The metal oxide functions as a barrier film between the insulating layer 124 and platinum, and the metal oxide contains at least one of titanium oxide (TiO2), chromium oxide (Cr2O3), tantalum pentoxide (Ta2O5), and oxygen-deficient metal oxide.

In the thermal-conduction microheater 10 configured as described above, the thin-film layer 12 generates heat according to power (hereinafter, also referred to as “heater power”) supplied from the control apparatus 100. The temperature of the thin-film layer 12 changes according to the thermal conductivity of the gas contained in the atmosphere provided with the microheater 10.

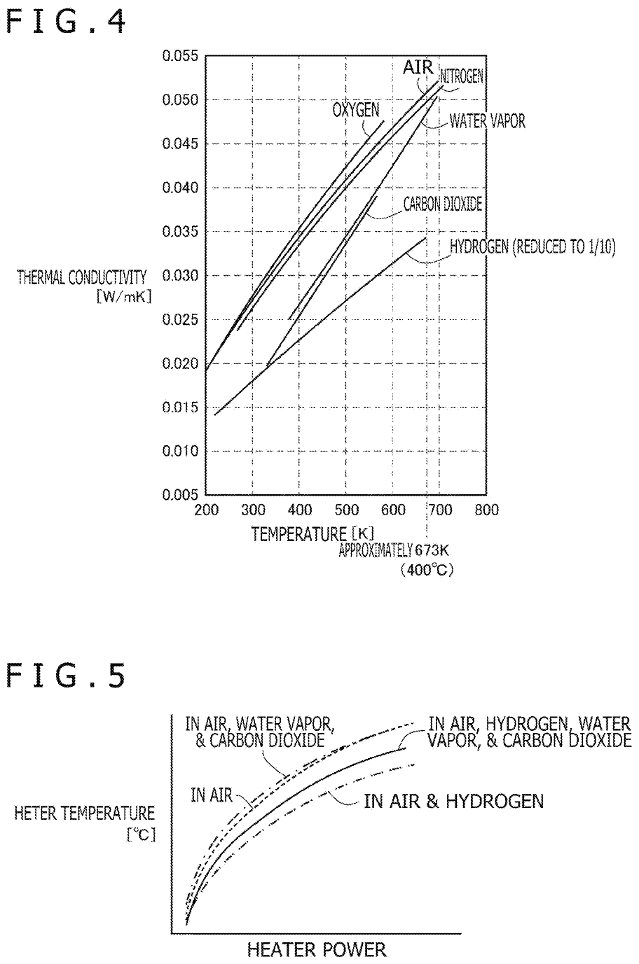

The thermal conductivity of each of a plurality of types of gases will be described here with reference to FIGS. 3 and 4. The thermal conductivity of hydrogen, oxygen, air, nitrogen, water vapor, and carbon dioxide is different from each other. FIG. 3 depicts an example of the thermal conductivity of each of the plurality of types of gases. As illustrated in FIG. 3, the thermal conductivity of hydrogen is the highest when the temperature of the atmosphere containing the gases is 0° C., followed by oxygen, air, and nitrogen in descending order of thermal conductivity, and water vapor and carbon dioxide have the lowest thermal conductivity. Note that the water vapor and carbon dioxide have the same or substantially the same thermal conductivity. The air is standard air and is, for example, air at the shipment of the microheater 10.

The thermal conductivity of each of the gases including hydrogen, oxygen, air, nitrogen, water vapor, and carbon dioxide changes according to the change in the temperature of the atmosphere containing the gases. FIG. 4 depicts an example of the thermal conductivity relative to the temperature of each of the plurality of types of gases. Note that the thermal conductivity of hydrogen is reduced to one tenth in the graph illustrated in FIG. 4.

As illustrated in FIG. 4, the thermal conductivity of each gas also changes according to the change in the temperature of the atmosphere between 200 K and 800 K. Specifically, the higher the temperature of the atmosphere, the higher the thermal conductivity of each gas. When the temperature of the atmosphere is lower than approximately 673 K (400° C.), the difference between the thermal conductivity of air and the thermal conductivity of water vapor and carbon dioxide is equal to or greater than a predetermined value (for example, 0.001 W/mk). On the other hand, when the temperature of the atmosphere is approximately 673 K (400° C.) or higher, the difference between the thermal conductivity of air and the thermal conductivity of water vapor and carbon dioxide is smaller than the predetermined value (for example, 0.001 W/mk), and the thermal conductivity of air and the thermal conductivity of water vapor and carbon dioxide are the same or substantially the same.

In this way, the thermal conductivity varies according to the type of gas contained in the atmosphere provided with the microheater 10. Therefore, the amount of generated heat of the thin-film layer 12 varies according to the type (thermal conductivity) of gas contained in the atmosphere. For example, the thermal conductivity of hydrogen is higher than the thermal conductivity of air, water vapor, and carbon dioxide. Therefore, when the gas contained in the atmosphere is hydrogen, the heat of the thin-film layer 12 is easily released into the atmosphere, and the temperature (hereinafter, also referred to as “heater temperature”) of the microheater 10 (thin-film layer 12) is low, compared to when the gas contained in the atmosphere is air, water vapor, or carbon dioxide. The thermal conductivity of water vapor and carbon dioxide is lower than the thermal conductivity of hydrogen and air. Therefore, when the gas contained in the atmosphere is water vapor or carbon dioxide, the heat of the thin-film layer 12 is not easily released into the atmosphere, and the heater temperature is high, compared to when the gas contained in the atmosphere is hydrogen or air.

That is, the heater temperature changes according to the type (thermal conductivity) of gas contained in the atmosphere provided with the microheater 10, and the resistance value of platinum of the sensor unit 13 changes according to the heater temperature. The control apparatus 100 can use such a phenomenon to acquire, as a detection value, the resistance value of platinum of the sensor unit 13 and measure the concentration of gas contained in the atmosphere based on the acquired detection value. Note that the control apparatus 100 may directly detect, as a detection value, the heater temperature of the thin-film layer 12.

FIG. 1 will be further described. The control apparatus 100 is configured to measure the concentration of gas contained in the atmosphere based on the detection value acquired from the microheater 10 (resistance value of platinum of the sensor unit 13). Specifically, the control apparatus 100 measures at least the concentration of hydrogen contained in the atmosphere provided with the microheater 10. The control apparatus 100 is an information processing apparatus, such as a notebook or desktop PC, a smartphone, a tablet terminal, an in-vehicle ECU (engine control unit), and a PLC (programmable logic controller) of a production facility.

Main components of the control apparatus 100 include an arithmetic unit 101, a memory 102, a storage apparatus 103, a communication apparatus 104, a display interface 105, a peripheral device interface 106, a storage medium interface 107, and a microheater interface 108.

The arithmetic unit 101 is a computation entity (computer) that executes various programs to execute various processes, and the arithmetic unit 101 is an example of a “calculation unit.” The arithmetic unit 101 includes a processor, such as a CPU (central processing unit) and an MPU (micro-processing unit). Although the processor as an example of the arithmetic unit 101 has functions of executing programs to execute various processes, some or all of the functions may be implemented by using a dedicated hardware circuit, such as an ASIC (application specific integrated circuit) and an FPGA (field-programmable gate array). The “processor” may include not only a processor in a narrow sense, such as a CPU and an MPU, that uses a stored-program system to execute processes, but may also include a hard-wired circuit, such as an ASIC and an FPGA. Therefore, the “processor” as an example of the arithmetic unit 101 may be referred to as processing circuitry, in which the process is defined in advance by a computer-readable code and/or a hard-wired circuit. Note that the arithmetic unit 101 may include one chip or may include a plurality of chips. A server apparatus not illustrated (for example, cloud server apparatus) may be provided with some of the functions of the arithmetic unit 101.

The memory 102 includes a volatile storage area (for example, working area) for temporarily storing a program code, a work memory, or the like when the arithmetic unit 101 executes various programs. Examples of the memory 102 include a volatile memory, such as a DRAM (dynamic random access memory) and a SRAM (static random access memory), and a non-volatile memory, such as a ROM (read only memory) and a flash memory.

The storage apparatus 103 stores various programs executed by the arithmetic unit 101, various types of data, and the like. The storage apparatus 103 may include one or a plurality of non-transitory computer readable media or may include one or a plurality of computer readable storage media. Examples of the storage apparatus 103 include an HDD (hard disk drive) and an SSD (solid state drive). In the control apparatus 100 of the embodiment, the storage apparatus 103 stores a control program 120 and thermal conductivity data 130.

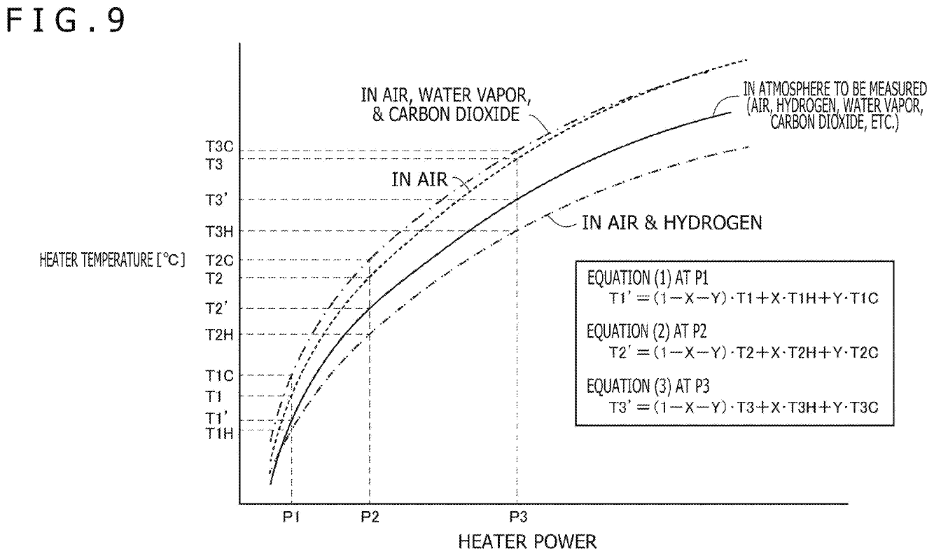

A process (hereinafter, also referred to as “concentration measurement process”) for measuring the concentration of at least the hydrogen contained in the atmosphere based on the detection value acquired by the arithmetic unit 101 from the microheater 10 is defined in the control program 120. The thermal conductivity data 130 includes data related to the thermal conductivity of each of a plurality of types of gases contained in a gas mixture. The thermal conductivity data 130 includes data representing the thermal conductivity relative to the temperature of each gas as illustrated in FIG. 4 and includes data representing the heater temperature relative to the heater power of each gas as illustrated in FIG. 9 described later.

The communication apparatus 104 uses wired communication or wireless communication to transmit and receive data to and from an external apparatus not illustrated.

The display interface 105 is an interface for connecting a display 51. The display interface 105 realizes input and output of data between the control apparatus 100 and the display 51. For example, the arithmetic unit 101 controls the display interface 105 to output various types of image data to the display 51. The display 51 displays the image data acquired from the display interface 105.

The peripheral device interface 106 is an interface for connecting a peripheral device 60, such as a keyboard and a mouse. The peripheral device interface 106 realizes input and output of data between the control apparatus 100 and the peripheral device 60. For example, the peripheral device interface 106 acquires various types of data, such as the control program 120 and the thermal conductivity data 130 input by the user by using the peripheral device 60.

The storage medium interface 107 is an interface for connecting a storage medium 70, such as a removable disk and a USB (universal serial bus) memory. The storage medium interface 107 reads various types of data, such as a program and data, stored in the storage medium 70, and writes various types of data to the storage medium 70. For example, the storage medium interface 107 reads various types of data, such as the control program 120 and the thermal conductivity data 130 stored in the storage medium 70, from the storage medium 70 and writes various types of data, such as the control program 120 and the thermal conductivity data 130 stored in the storage apparatus 103, to the storage medium 70.

The microheater interface 108 is an interface for connecting the microheater 10 and is an example of a “detection unit.” The microheater interface 108 detects a detection value corresponding to the heater temperature and acquires the detection value. The arithmetic unit 101 controls the microheater interface 108 to supply heater power to the microheater 10. Note that the microheater interface 108 may be divided into separate components including an interface for acquiring the detection value from the microheater 10 and an interface for supplying the heater power to the microheater 10.

[Heater Temperature when Atmosphere Contains Gas Mixture]

The heater temperature when the atmosphere contains a gas mixture will be described with reference to FIGS. 5 to 8. As described above, the control apparatus 100 of the first embodiment is configured to measure the concentration of hydrogen contained in the atmosphere provided with the microheater 10 based on the detection value acquired from the microheater 10 (resistance value of platinum of the sensor unit 13 corresponding to the heater temperature).

The atmosphere contains not only one type of gas, such as hydrogen, but also a gas mixture containing a plurality of types of gases, such as air, water vapor, and carbon dioxide. The thermal conductivity relative to the temperature of each gas contained in the atmosphere is different from each other as illustrated in FIGS. 3 and 4. Therefore, when the atmosphere provided with the microheater 10 contains the gas mixture, the heater temperature of the microheater 10 is influenced by the thermal conductivity of the hydrogen to be measured as well as the thermal conductivity of the other gases, such as air, water vapor, and carbon dioxide, contained in the gas mixture. Thus, the detection value acquired by the control apparatus 100 from the microheater 10 is also influenced by the thermal conductivity of the hydrogen to be measured as well as the thermal conductivity of the other gases, such as air, water vapor, and carbon dioxide, contained in the gas mixture.

The fact that the heater temperature relative to the heater power varies according to the types of gases contained in the gas mixture will be described with reference to FIGS. 5 to 7. FIGS. 5 to 7 depict the heater temperature relative to the heater power corresponding to the types of gases contained in the gas mixture.

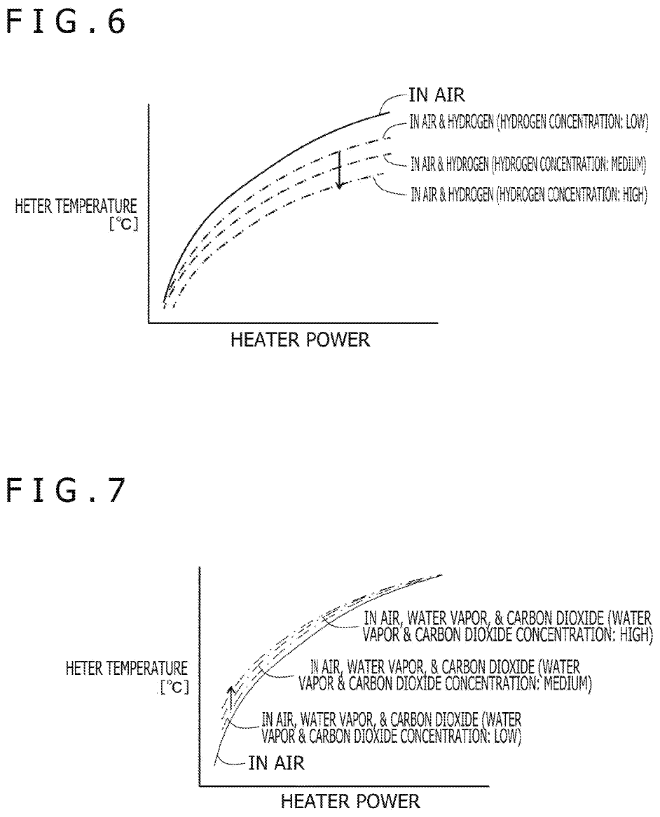

As illustrated in FIG. 5, when the air and hydrogen are the gases contained in the gas mixture, the heat of the thin-film layer 12 is easily released into the atmosphere due to the influence of the thermal conductivity of hydrogen, and the heater temperature relative to the heater power is low, compared to when only the air is the gas contained in the gas mixture. As illustrated in FIG. 6, the higher the concentration of hydron contained in the gas mixture, the lower the heater temperature relative to the heater power.

FIG. 5 will be further described. When the air, hydrogen, water vapor, and carbon dioxide are the gases contained in the gas mixture, the heat of the thin-film layer 12 is also easily released into the atmosphere due to the influence of the thermal conductivity of hydrogen, and the heater temperature relative to the heater power is also low, compared to when only the air is the gas contained in the gas mixture.

On the other hand, when the air, water vapor, and carbon dioxide are the gases contained in the gas mixture (that is, when hydrogen is not contained), the heat of the thin-film layer 12 is not easily released into the atmosphere due to the influence of the thermal conductivity of water vapor and carbon dioxide, and the heater temperature relative to the heater power is high, compared to when only the air is the gas contained in the gas mixture. As illustrated in FIG. 7, the higher the concentration of water vapor and carbon dioxide contained in the gas mixture, the higher the heater temperature relative to the heater power.

If, for example, a control apparatus of a comparative example calculates the hydrogen concentration based on the heater temperature without taking into account the concentration of the gases contained in the gas mixture, the hydrogen concentration may not be accurately measured in the atmosphere containing the gas mixture.

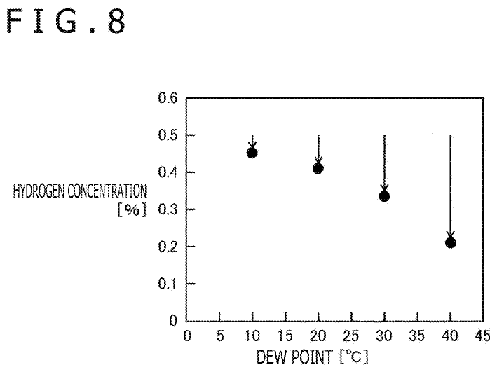

FIG. 8 depicts the hydrogen concentration relative to the dew point measured by the control apparatus of the comparative example. As illustrated in FIG. 8, the dew point of the atmosphere increases with an increase in the concentration of water vapor contained in the gas mixture in the atmosphere. The proportion of water vapor in the gas mixture increases when the concentration of water vapor contained in the gas mixture in the atmosphere increases while the concentration of hydrogen contained in the gas mixture is constant at 0.5%. This reduces the hydrogen concentration measured by the control apparatus of the comparative example. However, the concentration of hydrogen contained in the gas mixture is actually constant at 0.5%. In this way, the control apparatus of the comparative example calculates the hydrogen concentration based on the heater temperature without taking into account the concentration of the gas (water vapor in the example) other than the hydrogen contained in the gas mixture. Therefore, the hydrogen concentration may not be accurately measured.

Therefore, the control apparatus 100 of the first embodiment is configured to accurately measure the hydrogen concentration based on the detection value from the microheater 10 even in the atmosphere containing the gas mixture including other gases, such as air, water vapor, and carbon dioxide, in addition to the hydrogen to be measured.

### [Concentration Measurement Process by Control Apparatus]

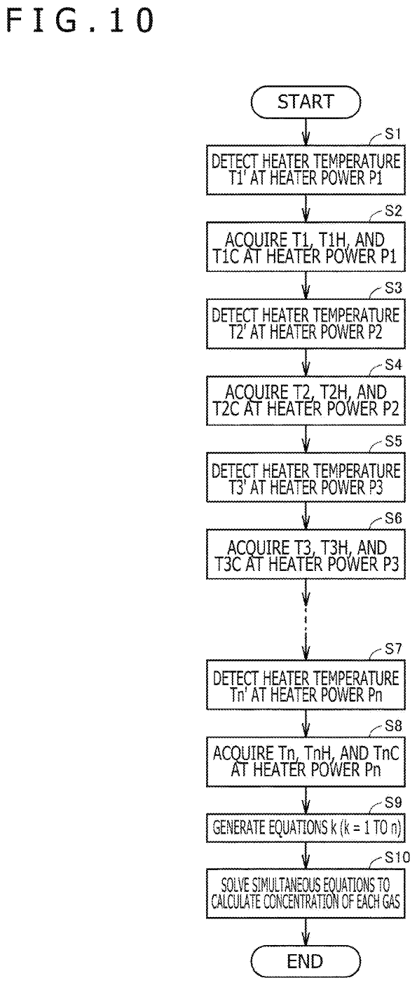

The concentration measurement process executed by the control apparatus 100 will be described with reference to FIGS. 9 and 10. FIG. 9 depicts an example of simultaneous equations used by the control apparatus 100 of the first embodiment to execute the concentration measurement process. A gas mixture of air, hydrogen, water vapor, and carbon dioxide is contained in the atmosphere to be measured provided with the microheater 10 in the example illustrated in FIG. 9.

The control apparatus 100 supplies varying heater power to the microheater 10 arranged in the atmosphere to be measured and acquires, from the microheater 10, the detection value relative to each level of heater power to thereby calculate data representing the heater temperature relative to the heater power as indicated by a solid line in FIG. 9. The control apparatus 100 can detect heater temperature T1′ as heater temperature at heater power P1, detect heater temperature T2′ as heater temperature at heater power P2, and detect heater temperature T3′ as heater temperature at heater power P3 based on the data.

The control apparatus 100 stores in advance, in the storage apparatus 103 and as the thermal conductivity data 130, data representing the heater temperature after varying and supplying heater power to the microheater 10 arranged in the atmosphere containing only the air. The control apparatus 100 stores in advance, in the storage apparatus 103 and as the thermal conductivity data 130, data representing the heater temperature after varying and supplying heater power to the microheater 10 arranged in the atmosphere containing a gas mixture of air and hydrogen. The control apparatus 100 stores in advance, in the storage apparatus 103 and as the thermal conductivity data 130, data representing the heater temperature after varying and supplying heater power to the microheater 10 arranged in the atmosphere containing a gas mixture of air, water vapor, and carbon dioxide.

Therefore, the control apparatus 100 can acquire heater temperature T1 as heater temperature at the heater power P1, acquire heater temperature T2 as heater temperature at the heater power P2, and acquire heater temperature T3 as heater temperature at the heater power P3 in relation to the microheater 10 arranged in the atmosphere containing only the air based on the thermal conductivity data 130. The control apparatus 100 can acquire heater temperature T1H as heater temperature at the heater power P1, acquire heater temperature T2H as heater temperature at the heater power P2, and acquire heater temperature T3H as heater temperature at the heater power P3 in relation to the microheater 10 arranged in the atmosphere containing the gas mixture of air and hydrogen based on the thermal conductivity data 130. The control apparatus 100 can acquire heater temperature TIC as heater temperature at the heater power P1, acquire heater temperature T2C as heater temperature at the heater power P2, and acquire heater temperature T3C as heater temperature at the heater power P3 in relation to the microheater 10 arranged in the atmosphere containing the gas mixture of air, water vapor, and carbon dioxide based on the thermal conductivity data 130.

The control apparatus 100 uses the heater temperature (T1′, T2′, or T3′) relative to the heater power (P1, P2, or P3) actually measured by the microheater 10 arranged in the atmosphere containing the gas mixture of air, hydrogen, water vapor, and carbon dioxide, the heater temperature (T1H, T2H, or T3H) relative to the heater power (P1, P2, or P3) of the gas mixture of air and hydrogen stored in advance, and the heater temperature (T1C, T2C, or T3C) relative to the heater power (P1, P2, or P3) of the gas mixture of air, water vapor, and carbon dioxide stored in advance to generate simultaneous equations including the following equations (1) to (3).

$$\begin{matrix}[ Math . 1 ] & \\ T {1}^{′} = ( 1 - X - Y ) \cdot T 1 + X \cdot T 1 H + Y \cdot T 1 C & ( 1 )\end{matrix}$$

In the simultaneous equations (1) to (3), T1′ represents the heater temperature at the time when the heater power P1 is supplied to the microheater 10 arranged in the atmosphere to be measured. T2′ represents the heater temperature at the time when the heater power P2 is supplied to the microheater 10 arranged in the atmosphere to be measured. T3′ represents the heater temperature at the time when the heater power P3 is supplied to the microheater 10 arranged in the atmosphere to be measured. T1 represents the heater temperature at the time when the heater power P1 is supplied to the microheater 10 arranged in the atmosphere containing only the air. T2 represents the heater temperature at the time when the heater power P2 is supplied to the microheater 10 arranged in the atmosphere containing only the air. T3 represents the heater temperature at the time when the heater power P3 is supplied to the microheater 10 arranged in the atmosphere containing only the air. T1H represents the heater temperature at the time when the heater power P1 is supplied to the microheater 10 arranged in the atmosphere containing the gas mixture of air and hydrogen. T2H represents the heater temperature at the time when the heater power P2 is supplied to the microheater 10 arranged in the atmosphere containing the gas mixture of air and hydrogen. T3H represents the heater temperature at the time when the heater power P3 is supplied to the microheater 10 arranged in the atmosphere containing the gas mixture of air and hydrogen. TIC represents the heater temperature at the time when the heater power P1 is supplied to the microheater 10 arranged in the atmosphere containing the gas mixture of air, water vapor, and carbon dioxide. T2C represents the heater temperature at the time when the heater power P2 is supplied to the microheater 10 arranged in the atmosphere containing the gas mixture of air, water vapor, and carbon dioxide. T3C represents the heater temperature at the time when the heater power P3 is supplied to the microheater 10 arranged in the atmosphere containing the gas mixture of air, water vapor, and carbon dioxide.

The control apparatus 100 does not know the concentration of each of the air, hydrogen, water vapor, and carbon dioxide in the gas mixture contained in the atmosphere to be measured. Therefore, characters, such as X and Y, are used to express the concentration of each gas. For example, X represents the concentration of hydrogen in the simultaneous equations (1) to (3). Y represents a total value of the concentration of water vapor and the concentration of carbon dioxide. 1-X-Y represents the concentration of air.

The control apparatus 100 can solve the simultaneous equations (1) to (3) to calculate X and Y. In this way, the control apparatus 100 can calculate the hydrogen concentration (X) and the total value (Y) of the water vapor concentration and the carbon dioxide concentration in the gas mixture contained in the atmosphere to be measured. Note that the hydrogen concentration (X) and the total value (Y) of the water vapor concentration and the carbon dioxide concentration can be calculated by using at least two equations. Therefore, the control apparatus 100 can use at least two of the equations (1) to (3) to solve the simultaneous equations.

The control apparatus 100 outputs the calculated hydrogen concentration according to the application in which the sensor module 1 is applied. For example, the control apparatus 100 may cause the display 51 to display an image representing the hydrogen concentration through the display interface 105. The control apparatus 100 may use the communication apparatus 104 to transmit data representing the hydrogen concentration to an external apparatus not illustrated. The control apparatus 100 may cause the storage medium 70 to store the data representing the hydrogen concentration through the storage medium interface 107. The control apparatus 100 may also control and operate an actuator not illustrated, according to the calculated hydrogen concentration.

FIG. 10 is a flow chart illustrating the concentration measurement process executed by the control apparatus 100 of the first embodiment. The flow chart illustrated in FIG. 10 defines steps of the concentration measurement process executed by the arithmetic unit 101 of the control apparatus 100 according to the control program 120. Note that “S” is used as an abbreviation of “STEP” in FIG. 10.

As illustrated in FIG. 10, the control apparatus 100 detects the heater temperature T1′ at the time when the heater power P1 is supplied to the microheater 10 (S1). The control apparatus 100 acquires the heater temperature T1 (heater temperature in the atmosphere containing only the air) at the heater power P1, the heater temperature T1H (heater temperature in the atmosphere containing the gas mixture of air and hydrogen) at the heater power P1, and the heater temperature TIC (heater temperature in the atmosphere containing the gas mixture of air, water vapor, and carbon dioxide) at the heater power P1 based on the thermal conductivity data 130 (S2).

The control apparatus 100 detects the heater temperature T2′ at the time when the heater power P2 is supplied to the microheater 10 (S3). The control apparatus 100 acquires the heater temperature T2 (heater temperature in the atmosphere containing only the air) at the heater power P2, the heater temperature T2H (heater temperature in the atmosphere containing the gas mixture of air and hydrogen) at the heater power P2, and the heater temperature T2C (heater temperature in the atmosphere containing the gas mixture of air, water vapor, and carbon dioxide) at the heater power P2 based on the thermal conductivity data 130 (S4).

The control apparatus 100 detects the heater temperature T3′ at the time when the heater power P3 is supplied to the microheater 10 (S5). The control apparatus 100 acquires the heater temperature T3 (heater temperature in the atmosphere containing only the air) at the heater power P3, the heater temperature T3H (heater temperature in the atmosphere containing the gas mixture of air and hydrogen) at the heater power P3, and the heater temperature T3C (heater temperature in the atmosphere containing the gas mixture of air, water vapor, and carbon dioxide) at the heater power P3 based on the thermal conductivity data 130 (S6).

The control apparatus 100 repeats the process to detect heater temperature Tn′ at the time when heater power Pn is supplied to the microheater 10 (S7). The control apparatus 100 acquires heater temperature Tn (heater temperature in the atmosphere containing only the air) at the heater power Pn, heater temperature TnH (heater temperature in the atmosphere containing the gas mixture of air and hydrogen) at the heater power Pn, and heater temperature TnC (heater temperature in the atmosphere containing the gas mixture of air, water vapor, and carbon dioxide) at the heater power Pn based on the thermal conductivity data 130 (S8).

The control apparatus 100 uses heater temperature Tk′, heater temperature Tk, heater temperature TkH, and heater temperature TkC to generate equations k (k=1 to n) like the equations (1) to (3) illustrated in FIG. 9. More specifically, the control apparatus 100 generates at least two equations like the equations (1) to (3) illustrated in FIG. 9 to generate simultaneous equations for calculating the hydrogen concentration (X) and the total value (Y) of the water vapor concentration and the carbon dioxide concentration. An equation (4) in the case of using the heater temperature Tk′, the heater temperature Tk, the heater temperature TkH, and the heater temperature TkC is as follows. Note that k represents natural numbers 1 to n in the equation (4). For example, the equation (4) is expressed by the equation (1) when k=1, and the equation (4) is expressed by the equation (2) when K=2.

$$\begin{matrix}[ Math . 2 ] & \\ T {2}^{′} = ( 1 - X - Y ) \cdot T 2 + X \cdot T 2 H + Y \cdot T 2 C & ( 2 )\end{matrix}$$

The control apparatus 100 solves the simultaneous equations to calculate the hydrogen concentration (X) and the total value (Y) of the water vapor concentration and the carbon dioxide concentration (S10). Subsequently, the control apparatus 100 ends the process.

In this way, the control apparatus 100 in the sensor module 1 of the first embodiment detects the heater temperature Tk′ at the time of the supply of heater power Pk and detects heater temperature Tk+1′ at the time of the supply of heater power Pk+1 higher than the heater power Pk. The control apparatus 100 calculates the hydrogen concentration (X) and the total value (Y) of the water vapor concentration and the carbon dioxide concentration contained in the gas mixture based on the heater temperature Tk′, the heater temperature Tk+1′, and the thermal conductivity data 130.

For example, the control apparatus 100 acquires the heater temperature T1 (heater temperature in the atmosphere containing only the air), the heater temperature T1H (heater temperature in the atmosphere containing the gas mixture of air and hydrogen), and the heater temperature TIC (heater temperature in the atmosphere containing the gas mixture of air, water vapor, and carbon dioxide) at the time of the supply of the heater power P1 based on the thermal conductivity data 130 and generates the equation (1) illustrated in FIG. 9 based on the heater temperature T1, the heater temperature T1H, the heater temperature TIC, and the heater temperature T1′. The control apparatus 100 acquires the heater temperature T2 (heater temperature in the atmosphere containing only the air), the heater temperature T2H (heater temperature in the atmosphere containing the gas mixture of air and hydrogen), and the heater temperature T2C (heater temperature in the atmosphere containing the gas mixture of air, water vapor, and carbon dioxide) at the time of the supply of the heater power P2 based on the thermal conductivity data 130 and generates the equation (2) illustrated in FIG. 9 based on the heater temperature T2, the heater temperature T2H, the heater temperature T2C, and the heater temperature T2′. The control apparatus 100 solves the simultaneous equations including the equation (1) and the equation (2) to calculate the hydrogen concentration (X) and the total value (Y) of the water vapor concentration and the carbon dioxide concentration contained in the gas mixture.

In this way, the control apparatus 100 can accurately measure the hydrogen concentration (X) based on the detection value from the microheater 10 even in the atmosphere containing the gas mixture including other gasses, such as air, water vapor, and carbon dioxide, in addition to the hydrogen to be measured.

Note that the control apparatus 100 can generate the simultaneous equations by using two or more of the equations with natural numbers k=1 to n in the equation (4). The control apparatus 100 may use average values of the heater temperature Tk′, the heater temperature Tk, the heater temperature TkH, and the heater temperature TkC in a plurality of equations to generate one equation. For example, the control apparatus 100 may apply, to the equation (4), an average value of the heater temperature T1′ and the heater temperature T2′, an average value of the heater temperature T1 and the heater temperature T2, an average value of the heater temperature T1H and the heater temperature T2H, and an average value of the heater temperature T1C and the heater temperature T2C to generate one equation.

### Second Embodiment

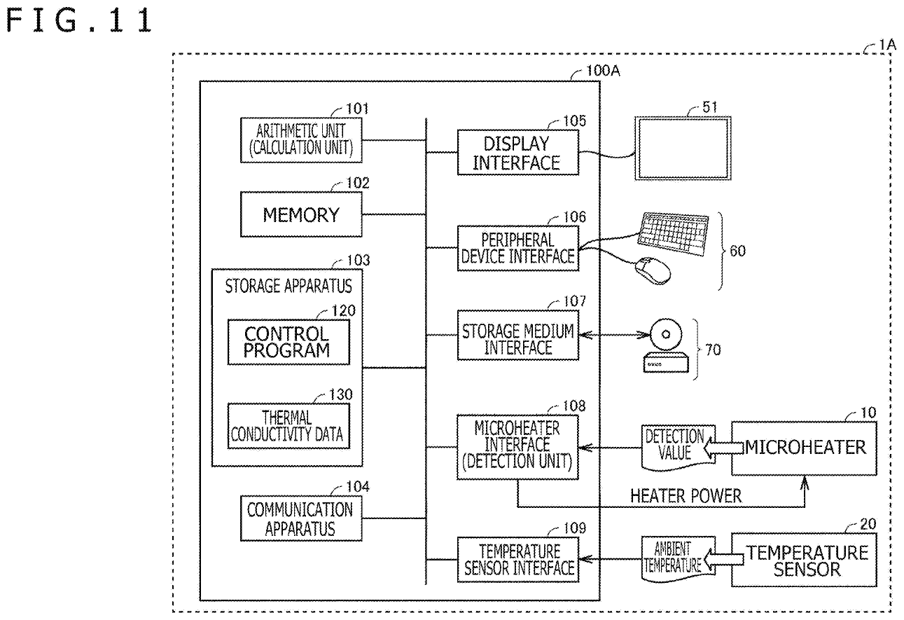

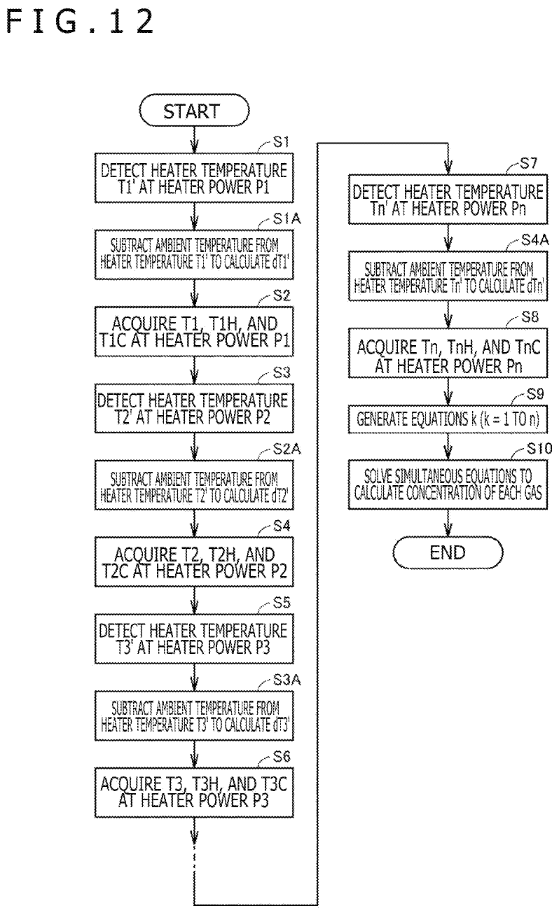

This completes the description of the sensor module 1 and the control apparatus 100 according to the first embodiment. The sensor module 1 and the control apparatus 100 may have other components and functions. A sensor module 1A and a control apparatus 100A of a second embodiment will be described with reference to FIGS. 11 and 12. Note that only the components and functions of the sensor module 1A and the control apparatus 100A of the second embodiment different from those of the sensor module 1 and the control apparatus 100 of the first embodiment will be described.

FIG. 11 is a diagram for describing the components of the sensor module 1A and the control apparatus 100A according to the second embodiment. The sensor module 1A further includes a temperature sensor 20 that measures ambient temperature (for example, room temperature) in the atmosphere provided with the microheater 10. The control apparatus 100A further includes a temperature sensor interface 109 that acquires the ambient temperature measured by the temperature sensor 20.

FIG. 12 is a flow chart illustrating a process of the control apparatus 100A according to the second embodiment. The flow chart illustrated in FIG. 12 defines steps of the concentration measurement process executed by the arithmetic unit 101 of the control apparatus 100A according to the control program 120. Note that “S” is used as an abbreviation of “STEP” in FIG. 12.

As illustrated in FIG. 12, the control apparatus 100A detects the heater temperature T1′ at the time when the heater power P1 is supplied to the microheater 10 in S1 and then subtracts the ambient temperature measured by the temperature sensor 20 from the detected heater temperature T1′ to calculate a temperature difference dT1′ (SIA). The control apparatus 100A detects the heater temperature T2′ at the time when the heater power P2 is supplied to the microheater 10 in S3 and then subtracts the ambient temperature measured by the temperature sensor 20 from the detected heater temperature T2′ to calculate a temperature difference dT2′ (S2A). The control apparatus 100A detects the heater temperature T3′ at the time when the heater power P3 is supplied to the microheater 10 in S5 and then subtracts the ambient temperature measured by the temperature sensor 20 from the detected heater temperature T3′ to calculate a temperature difference dT3′ (S3A).

The control apparatus 100A repeats the process to detect the heater temperature Tn′ at the time when the heater power Pn is supplied to the microheater 10 and then subtracts the ambient temperature measured by the temperature sensor 20 from the detected heater temperature Tn′ to calculate a temperature difference dTn′ (S4A).

The control apparatus 100A uses a temperature difference dTk′ instead of the heater temperature Tk′ in the equation (4) to generate equations k (k=1 to n) (S9). More specifically, the control apparatus 100A uses the temperature difference dTk′ instead of the heater temperature Tk′ to generate at least two equations to thereby generate simultaneous equations for calculating the hydrogen concentration (X) and the total value (Y) of the water vapor concentration and the carbon dioxide concentration. Note that an equation (5) in the case of using the temperature difference dTk′ instead of the heater temperature Tk′ is as follows. In the equation (5), k represents natural numbers 1 to n. For example, the equation (5) is expressed by an equation (6) when k=1, and the equation (5) is expressed by an equation (7) when k=2.

$$\begin{matrix}[ Math . 3 ] & \\ T {3}^{′} = ( 1 - X - Y ) \cdot T 3 + X \cdot T 3 H + Y \cdot T 3 C & ( 3 )\end{matrix}$$

The control apparatus 100A solves the simultaneous equations to calculate the hydrogen concentration (X) and the total value (Y) of the water vapor concentration and the carbon dioxide concentration (S10). Subsequently, the control apparatus 100A ends the process.

In this way, the control apparatus 100A in the sensor module 1A of the second embodiment subtracts the ambient temperature in the atmosphere provided with the microheater 10 from the heater temperature Tk′ at the time of the supply of the heater power Pk to calculate the temperature difference dTk′ and calculates the hydrogen concentration (X) and the total value (Y) of the water vapor concentration and the carbon dioxide concentration contained in the gas mixture based on the heater temperature Tk, the heater temperature TkH, the heater temperature TkC, and the heater temperature dTk′.

For example, the control apparatus 100A subtracts the ambient temperature in the atmosphere provided with the microheater 10 from the heater temperature T1′ at the time of the supply of the heater power P1 to calculate the temperature difference dT1′ and generates the equation (6) based on the heater temperature T1, the heater temperature T1H, the heater temperature TIC, and the heater temperature dT1′. The control apparatus 100A subtracts the ambient temperature in the atmosphere provided with the microheater 10 from the heater temperature T2′ at the time of the supply of the heater power P2 to calculate the temperature difference dT2′ and generates the equation (7) based on the heater temperature T2, the heater temperature T2H, the heater temperature T2C, and the heater temperature dT2′. The control apparatus 100A solves the simultaneous equations including the equation (6) and the equation (7) to calculate the hydrogen concentration (X) and the total value (Y) of the water vapor concentration and the carbon dioxide concentration contained in the gas mixture.

In this way, the control apparatus 100A can calculate the temperature difference dTk′ by subtracting the ambient temperature measured by the temperature sensor 20 at the time of the detection of the heater temperature Tk from the heater temperature Tk detected while changing the heater power Pk, such as the heater power P1 and the heater power P2, and use the calculated temperature different dTk′ to generate the simultaneous equations. This can eliminate the influence of the ambient temperature in the simultaneous equations for calculating the hydrogen concentration (X) and the total value (Y) of the water vapor concentration and the carbon dioxide concentration. As a result, the control apparatus 100A can more accurately measure the hydrogen concentration (X) based on the detection value from the microheater 10.

Note that the control apparatus 100A can generate the simultaneous equations by using two or more of the equations with natural numbers k=1 to n in the equation (5). The control apparatus 100A may use average values of the temperature difference dTk′, the heater temperature Tk, the heater temperature TkH, and the heater temperature TkC in a plurality of equations to generate one equation. For example, the control apparatus 100A may apply, to the equation (5), an average value of the temperature difference dT1′ and the temperature difference dT2′, an average value of the heater temperature T1 and the heater temperature T2, an average value of the heater temperature T1H and the heater temperature T2H, and an average value of the heater temperature TIC and the heater temperature T2C to generate one equation.

The thermal conductivity of air and the thermal conductivity of water vapor and carbon dioxide are the same or substantially the same when the temperature of the atmosphere is approximately 673 K (400° C.) or higher as illustrated in FIG. 4. Therefore, it is preferable that at least one of the temperature differences dTk′ in the simultaneous equations generated by using the equation (5) be approximately 673 K (400° C.) or more. For example, it is preferable that at least one of the temperature difference dT1′ used in the equation (6) and the temperature difference dT2′ used in the equation (7) be approximately 673 K (400° C.) or more. In this way, the concentration (1-X-Y) of air and the total value (Y) of the water vapor concentration and the carbon dioxide concentration can be handled as the same value in at least one of the equation (6) and the equation (7). Therefore, the simultaneous equations can be simplified, and the concentration measurement process can be more efficiently executed.

### Third Embodiment

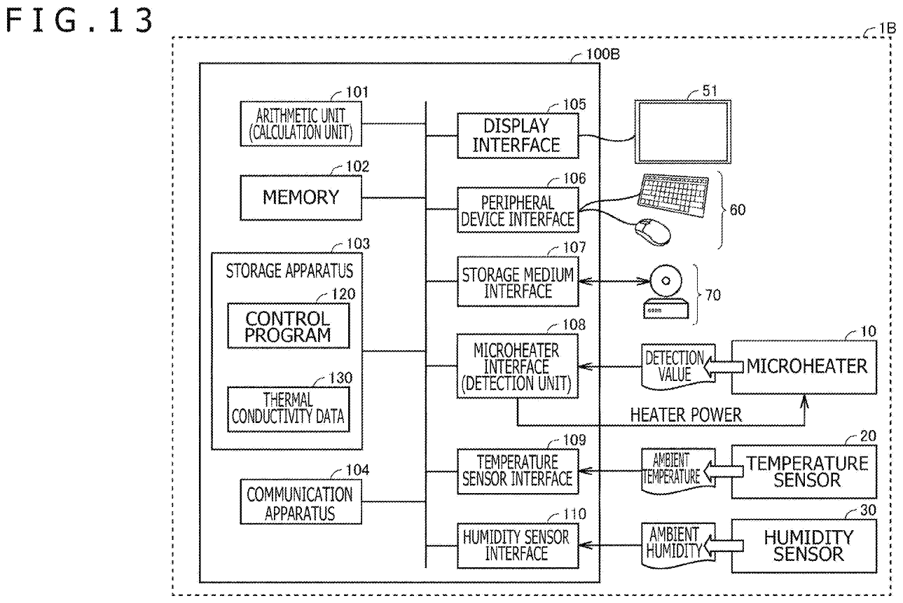

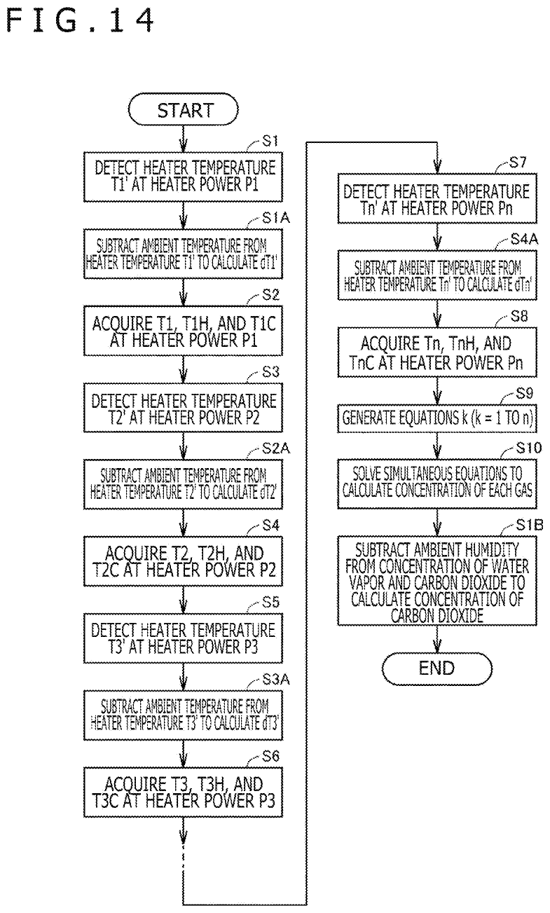

A sensor module 1B and a control apparatus 100B of a third embodiment will be described with reference to FIGS. 13 and 14. Note that only the components and functions of the sensor module 1B and the control apparatus 100B of the third embodiment different from those of the sensor module 1A and the control apparatus 100A of the second embodiment will be described.

FIG. 13 is a diagram for describing the components of the sensor module 1B and the control apparatus 100B according to the third embodiment. The sensor module 1B further includes a humidity sensor 30 that measures ambient humidity (for example, room humidity) in the atmosphere provided with the microheater 10. The control apparatus 100B further includes a humidity sensor interface 110 that acquires the ambient humidity measured by the humidity sensor 30.

FIG. 14 is a flow chart illustrating a process of the control apparatus 100B according to the third embodiment. The flow chart illustrated in FIG. 14 defines steps of the concentration measurement process executed by the arithmetic unit 101 of the control apparatus 100B according to the control program 120. Note that “S” is used as an abbreviation of “STEP” in FIG. 14.

As illustrated in FIG. 14, the control apparatus 100B solves the simultaneous equations to calculate the hydrogen concentration (X) and the total value (Y) of the water vapor concentration and the carbon dioxide concentration in S10 and then subtracts the ambient humidity measured by the humidity sensor 30 from the calculated total value (Y) of the water vapor concentration and the carbon dioxide concentration to calculate the carbon dioxide concentration (S1B).

In this way, the control apparatus 100B in the sensor module 1B of the third embodiment can use the humidity sensor 30 to directly measure the water vapor concentration and subtract the measured water vapor concentration from the total value (Y) of the water vapor concentration and the carbon dioxide concentration to thereby calculate the carbon dioxide concentration. In this way, the control apparatus 100B can separately measure the hydrogen concentration, the water vapor concentration, and the carbon dioxide concentration.

Note that the process of SIB executed by the control apparatus 100B of the third embodiment may be executed by the control apparatus 100 of the first embodiment after S10.

### <Supplement>

### (Item 1)

Sensor modules 1, 1A, and 1B include a microheater 10 of thermal conduction type that generates heat according to supplied power, and the control apparatuses 100, 100A, and 100B that are able to communicate with the microheater 10. The control apparatuses 100, 100A, and 100B detect a first detection value corresponding to first temperature of the microheater 10 at a time of supply of first power, detect a second detection value corresponding to second temperature of the microheater 10 at a time of supply of second power higher than the first power, and calculate the concentration of the at least one type of gas contained in the gas mixture based on the first detection value, the second detection value, and thermal conductivity data 130 related to thermal conductivity of each of a plurality of types of gases contained in the gas mixture.

According to the configuration, the control apparatuses 100, 100A, and 100B can accurately measure the concentration of the at least one type of gas contained in the gas mixture based on the detection value from the microheater 10.

### (Item 2)

In the sensor modules 1, 1A, and 1B according to Item 1, the control apparatuses 100, 100A, and 100B acquire temperature of each of the plurality of types of gases at the time when the first power is supplied to the microheater 10 based on the thermal conductivity data 130 to generate a first equation for calculating the concentration of the at least one type of gas based on the acquired temperature of each of the plurality of types of gases and the first temperature corresponding to the first detection value, acquire temperature of each of the plurality of types of gases at the time when the second power is supplied to the microheater 10 based on the thermal conductivity data 130 to generate a second equation for calculating the concentration of the at least one type of gas based on the acquired temperature of each of the plurality of gases and the second temperature corresponding to the second detection value, and calculate the concentration of the at least one type of gas based on the first equation and the second equation.

According to the configuration, the control apparatuses 100, 100A, and 100B can use the simultaneous equations including the first equation and the second equation to accurately measure the concentration of at least one type of gas contained in the gas mixture.

### (Item 3)

In the sensor modules 1A and 1B according to Item 1 or 2, the control apparatuses 100A and 100B subtracts temperature of atmosphere provided with the microheater 10 from the first temperature corresponding to the first detection value to calculate a first temperature difference, generates the first equation based on the temperature of each of the plurality of types of gases at the time when the first power is supplied to the microheater 10 and the first temperature difference, subtracts the temperature of the atmosphere from the second temperature corresponding to the second detection value to calculate a second temperature difference, and generates the second equation based on the temperature of each of the plurality of types of gases at the time when the second power is supplied to the microheater 10 and the second temperature difference.

According to the configuration, the control apparatuses 100A and 100B can eliminate the influence of the temperature of the atmosphere in the simultaneous equations including the first equation and the second equation. Therefore, the control apparatuses 100A and 100B can more accurately measure the concentration of at least one type of gas contained in the gas mixture.

### (Item 4)

In the sensor modules 1A and 1B according to Item 3, at least one of the first temperature difference and the second temperature difference is 400° C. or more.

According to the configuration, the control apparatuses 100A and 100B can simplify the simultaneous equations and efficiently execute the process for measuring the concentration of at least one type of gas.

### (Item 5)

In the sensor modules 1, 1A, and 1B according to any one of Items 1 to 4, the at least one type of gas contains hydrogen.

According to the configuration, the control apparatuses 100, 100A, and 100B can accurately measure the concentration of hydrogen contained in the gas mixture.

### (Item 6)

The sensor module 1B according to any one of Items 1 to 5 further includes a humidity sensor 30 that detects humidity of atmosphere provided with the microheater 10. The at least one type of gas contains carbon dioxide and water vapor. The control apparatus 100B subtracts the humidity of the atmosphere provided with the microheater 10 from the concentration of the at least one type of gas to calculate the concentration of the carbon dioxide.

According to the configuration, the control apparatus 100B can separately measure the concentration of water vapor and the concentration of carbon dioxide contained in the gas mixture.

### (Item 7)

In the sensor modules 1, 1A, and 1B according to any one of Items 1 to 6, the microheater 10 contains an insulating layer, a metal oxide provided on the insulating layer, and platinum provided on the metal oxide.

According to the configuration, the control apparatuses 100, 100A, and 100B can acquire the detection value from the microheater 10 even when the temperature of the atmosphere is approximately 673 K (400° C.) or higher.

### (Item 8)

In the sensor modules 1, 1A, and 1B according to Item 7, the metal oxide contains at least one of titanium oxide, chromium oxide, tantalum pentoxide, and oxygen-deficient metal oxide.

According to the configuration, the microheater 10 can appropriately generate heat according to the thermal conductivity of the gas mixture contained in the atmosphere.

### (Item 9)

Control apparatuses 100, 100A, and 100B include a detection unit (microheater interface 108) that detects a detection value corresponding to temperature of a microheater 10 of thermal conduction type that generates heat according to supplied power, and a calculation unit (arithmetic unit 101) that calculates the concentration of the at least one type of gas contained in the gas mixture based on the detection value detected by the detection unit. The calculation unit acquires a first detection value detected by the detection unit and corresponding to first temperature of the microheater 10 at a time of supply of first power, acquires a second detection value detected by the detection unit and corresponding to second temperature of the microheater 10 at a time of supply of second power higher than the first power, and calculates the concentration of the at least one type of gas contained in the gas mixture based on the first detection value, the second detection value, and thermal conductivity data 130 related to thermal conductivity of each of a plurality of types of gases contained in the gas mixture.

According to the configuration, the control apparatuses 100, 100A, and 100B can accurately measure the concentration of the at least one type of gas contained in the gas mixture based on the detection value from the microheater 10 even in the atmosphere containing the gas mixture.

### (Item 10)

A control method for controlling control apparatuses 100, 100A, and 100B includes a step (S1) of detecting a first detection value corresponding to first temperature of a microheater 10 of thermal conduction type at the time when first power is supplied to the microheater 10 that generates heat according to supplied power, a step (S3) of detecting a second detection value corresponding to second temperature of the microheater 10 at the time when second power higher than the first power is supplied to the microheater 10, and steps (S8 to S10) of calculating the concentration of the at least one type of gas contained in the gas mixture based on the first detection value, the second detection value, and thermal conductivity data 130 related to thermal conductivity of each of a plurality of types of gases contained in the gas mixture.

According to the configuration, the control apparatuses 100, 100A, and 100B can accurately measure the concentration of the at least one type of gas contained in the gas mixture based on the detection value from the microheater 10 even in the atmosphere containing the gas mixture.

### (Item 11)

A control program 120 causes a control apparatus 100 to execute a step (S1) of detecting a first detection value corresponding to first temperature of a microheater 10 of thermal conduction type at the time when first power is supplied to the microheater 10 that generates heat according to supplied power, a step (S3) of detecting a second detection value corresponding to second temperature of the microheater 10 at the time when second power higher than the first power is supplied to the microheater 10, and steps (S8 to S10) of calculating the concentration of the at least one type of gas contained in the gas mixture based on the first detection value, the second detection value, and thermal conductivity data 130 related to thermal conductivity of each of a plurality of types of gases contained in the gas mixture.

According to the configuration, the control apparatuses 100, 100A, and 100B can accurately measure the concentration of the at least one type of gas contained in the gas mixture based on the detection value from the microheater 10 even in the atmosphere containing the gas mixture.

The embodiments disclosed this time are illustrative in all aspects and should not be construed as restrictive. The scope of the present disclosure is indicated by the claims rather than the description of the embodiments, and all changes within the meaning and range of equivalents of the claims are intended to be included in the scope of the present disclosure.

According to the present disclosure, the concentration of the gas to be measured can be accurately measured in the atmosphere containing the gas mixture.

What is claimed is:

1. A sensor module that measures concentration of at least one type of gas contained in a gas mixture, the sensor module comprising:
a microheater of thermal conduction type that generates heat according to supplied power; and
a control apparatus that is able to communicate with the microheater, wherein
the control apparatus
detects a first detection value corresponding to first temperature of the microheater at a time of supply of first power,
detects a second detection value corresponding to second temperature of the microheater at a time of supply of second power higher than the first power, and
calculates the concentration of the at least one type of gas contained in the gas mixture based on the first detection value, the second detection value, and thermal conductivity data related to thermal conductivity of each of a plurality of types of gases contained in the gas mixture.

2. The sensor module according to claim 1, wherein
the control apparatus
acquires temperature of each of the plurality of types of gases at a time when the first power is supplied to the microheater based on the thermal conductivity data to generate a first equation for calculating the concentration of the at least one type of gas based on the acquired temperature of each of the plurality of types of gases and the first temperature corresponding to the first detection value,
acquires temperature of each of the plurality of types of gases at a time when the second power is supplied to the microheater based on the thermal conductivity data to generate a second equation for calculating the concentration of the at least one type of gas based on the acquired temperature of each of the plurality of gases and the second temperature corresponding to the second detection value, and
calculates the concentration of the at least one type of gas based on the first equation and the second equation.

3. The sensor module according to claim 2, wherein
the control apparatus
subtracts temperature of atmosphere provided with the microheater from the first temperature corresponding to the first detection value to calculate a first temperature difference,
generates the first equation based on the temperature of each of the plurality of types of gases at the time when the first power is supplied to the microheater and the first temperature difference,
subtracts the temperature of the atmosphere from the second temperature corresponding to the second detection value to calculate a second temperature difference, and
generates the second equation based on the temperature of each of the plurality of types of gases at the time when the second power is supplied to the microheater and the second temperature difference.

4. The sensor module according to claim 3, wherein
at least one of the first temperature difference and the second temperature difference is 400° C. or more.

5. The sensor module according to claim 1, wherein
the at least one type of gas contains hydrogen.

6. The sensor module according to claim 1, further comprising:
a humidity sensor that detects humidity of atmosphere provided with the microheater, wherein
the at least one type of gas contains carbon dioxide and water vapor, and
the control apparatus subtracts the humidity of the atmosphere provided with the microheater from the concentration of the at least one type of gas to calculate the concentration of the carbon dioxide.

7. The sensor module according to claim 1, wherein
the microheater contains an insulating layer, a metal oxide provided on the insulating layer, and platinum provided on the metal oxide.

8. The sensor module according to claim 7, wherein
the metal oxide contains at least one of titanium oxide, chromium oxide, tantalum pentoxide, and oxygen-deficient metal oxide.

9. A control apparatus that measures concentration of at least one type of gas contained in a gas mixture, the control apparatus comprising:
a detection unit that detects a detection value corresponding to temperature of a microheater of thermal conduction type that generates heat according to supplied power; and
a calculation unit that calculates the concentration of the at least one type of gas contained in the gas mixture based on the detection value detected by the detection unit, wherein
the calculation unit
acquires a first detection value detected by the detection unit and corresponding to first temperature of the microheater at a time of supply of first power,
acquires a second detection value detected by the detection unit and corresponding to second temperature of the microheater at a time of supply of second power higher than the first power, and
calculates the concentration of the at least one type of gas contained in the gas mixture based on the first detection value, the second detection value, and thermal conductivity data related to thermal conductivity of each of a plurality of types of gases contained in the gas mixture.

10. A control method for controlling a control apparatus that measures concentration of at least one type of gas contained in a gas mixture, the control method comprising:
detecting a first detection value corresponding to first temperature of a microheater of thermal conduction type at a time when first power is supplied to the microheater that generates heat according to supplied power;
detecting a second detection value corresponding to second temperature of the microheater at a time when second power higher than the first power is supplied to the microheater; and
calculating the concentration of the at least one type of gas contained in the gas mixture based on the first detection value, the second detection value, and thermal conductivity data related to thermal conductivity of each of a plurality of types of gases contained in the gas mixture.

11. A control program for measuring concentration of at least one type of gas contained in a gas mixture, the control program causing a control apparatus to execute:
a step of detecting a first detection value corresponding to first temperature of a microheater of thermal conduction type at a time when first power is supplied to the microheater that generates heat according to supplied power;
a step of detecting a second detection value corresponding to second temperature of the microheater at a time when second power higher than the first power is supplied to the microheater; and
a step of calculating the concentration of the at least one type of gas contained in the gas mixture based on the first detection value, the second detection value, and thermal conductivity data related to thermal conductivity of each of a plurality of types of gases contained in the gas mixture.
